(19) United States
(12) Patent Application Publication
(10) Pub. No.: US 2021/0213449 A1
(43) Pub. Date: Jul. 15, 2021

(54) DIELECTROPHORESIS AND DENSITY SEPARATORS

(71) Applicant: Hewlett-Packard Development Company, L.P., Spring, TX (US)

(72) Inventors: Viktor Shkolnikov, Palo Aito, CA (US); Daixi Xin, Palo Alto, CA (US); Chien-Hua Chen, Corvallis, OR (US)

(73) Assignee: Hewlett-Packard Development Company, L.P., Spring, TX (US)

(21) Appl. No.: 17/054,499

(22) PCT Filed: Sep. 26, 2018
(86) PCT No.: PCT/US2018/052930

§ 371 Date: Nov. 10, 2020

(51) Int. Cl. B01L 3/00; B01D 57/02; G01N 1/28; G01N 33/49; G01N 35/08

(52) U.S. Cl. CPC B01L 3/502715; B01D 57/02; B01L 3/50215; G01N 33/49; G01N 35/08; G01N 1/28

(57) ABSTRACT

In example implementations, an apparatus is provided. The apparatus includes a microfluidic channel to receive a fluid containing a plurality of different cells. A dielectrophoresis (DEP) separator in the apparatus separates the plurality of different cells passing through DEP separator within the microfluidic channel. In addition, the apparatus includes a density separator to further separate a portion of the plurality of different cells from the DEP separator based on a density of each one of the plurality of different cells.

### BACKGROUND

Various different industries use different devices to perform particle separation. In the medical industry, separation devices can be used to separate cells or other particles in solution for various different applications. For example, separation devices can be used to extract rare particles out of a mixture of common particles (e.g., where a ratio of common to rare is <1:1000). One example of such a separation may be to separate tumor cells from other cells in the blood of a patient. The separated tumor cells can then be used for diagnosis or otherwise be analyzed.

### BRIEF DESCRIPTION OF THE DRAWINGS

FIG. 1 is a block diagram of an example apparatus that performs dielectrophoresis (DEP) and density separation of the present disclosure;

FIG. 2 is a top view of an example of a DEP and density separator device of the present disclosure;

FIG. 3 is a side view of density separator portion of the DEP and density separator device;

FIG. 4 is a schematic diagram of a vertical separation of particles in the density separator portion of the DEP and density separator device;

FIG. 5 is a flow chart of an example method for separating particle.

### DETAILED DESCRIPTION

Examples described herein provide a microfluidic device that can separate particles in a fluid. For example, the microfluidic device can be a continuous DEP and density separator that can separate particles based on DEP and density. As discussed above, separation of particles in the medical industry can have important applications, such as separating out tumor cells in a patient's blood.

In some examples, microfluidic devices use planar electrodes that can generate non-uniform force fields. As a result, continuous separation of particles can be poor as two particles with the exact same properties traveling through the device can experience two different force field strengths.

Examples herein provide a microfluidic device that controls the location of the particles such that the particles experience the same force field strength. Thus, the separation of the particles based on size and polarization properties via DEP is consistent.

In addition, the microfluidic device of the present disclosure provides an additional level of separation based on density of the particles. For example, a first separation may be performed in a horizontal direction via DEP and a second separation may be performed in a vertical direction based on density. Thus, the microfluidic device of the present disclosure provides a DEP and density based separator that can consistently and accurately separate particles that have a high rare to common ratio.

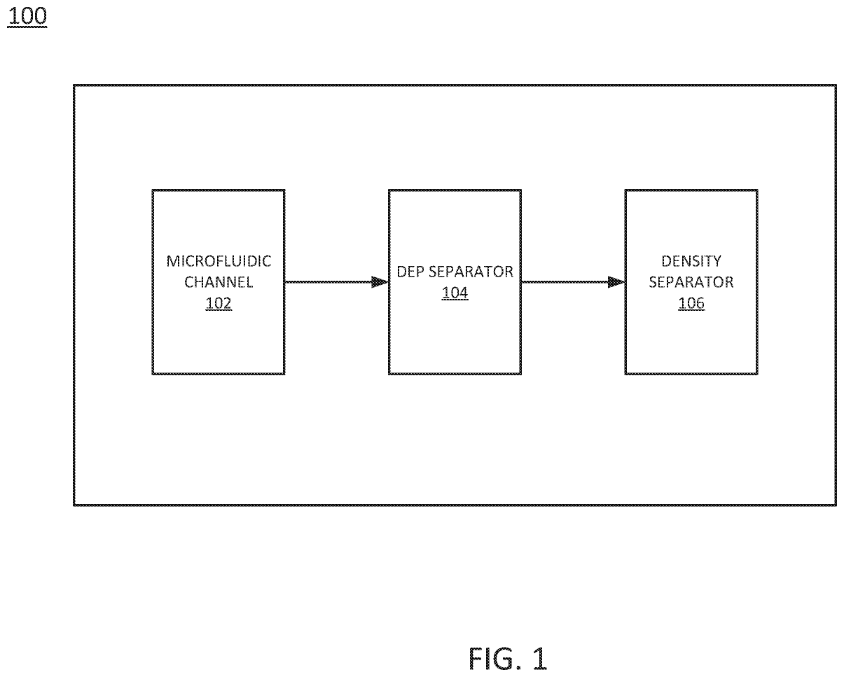

FIG. 1 illustrates an example microfluidic device 100. In one example, the microfluidic device 100 includes a microfluidic channel 102, a DEP separator 104, and a density separator 106. In one example, the microfluidic channel 102 may include a plurality of different microfluidic channels 102 that are combined into a single stream via a focusing region (discussed below). The microfluidic channel 102 may receive a fluid containing a plurality of different cells that are to be separated by the microfluidic device 100.

In one example, the fluid may be injected into the microfluidic channel 102 and flow towards the DEP separator 104. The DEP separator 104 may be a region in the microfluidic device 100 including components that create an electric field. The electric field causes the different types of cells to move towards one side of the microfluidic channel 102 or the other based on the dielectrophoresis properties or polarity of each type of cell.

In one example, the DEP separator 104 may separate the cells in a horizontal direction or along a horizontal plane. In other words, the DEP separator 104 may separate the cells, via the electric field, in a left and right direction along a horizontal plane (e.g., a plane that is parallel to the DEP separator 104).

In one example, one of the channels that is separated from the DEP separator 104 may continue to flow towards the density separator 106. In one example, the density separator 106 may further separate the fluid, or at least a portion of the fluid from the DEP separator 104, into the different types of cells that are in the fluid. In one example, the density separator 106 may be downstream from the DEP separator 104.

In one example, the density separator 106 may separate the cells based on the density of each type of cell. For example, a first cell may have a higher density than a second cell. As a result, the first cell may “sink” in the fluid as the fluid flows towards the density separator 106. The density separator 106 may separate the cells in a vertical direction. In other words, the density separator 106 may separate the cells in an up and down direction along a vertical plane.

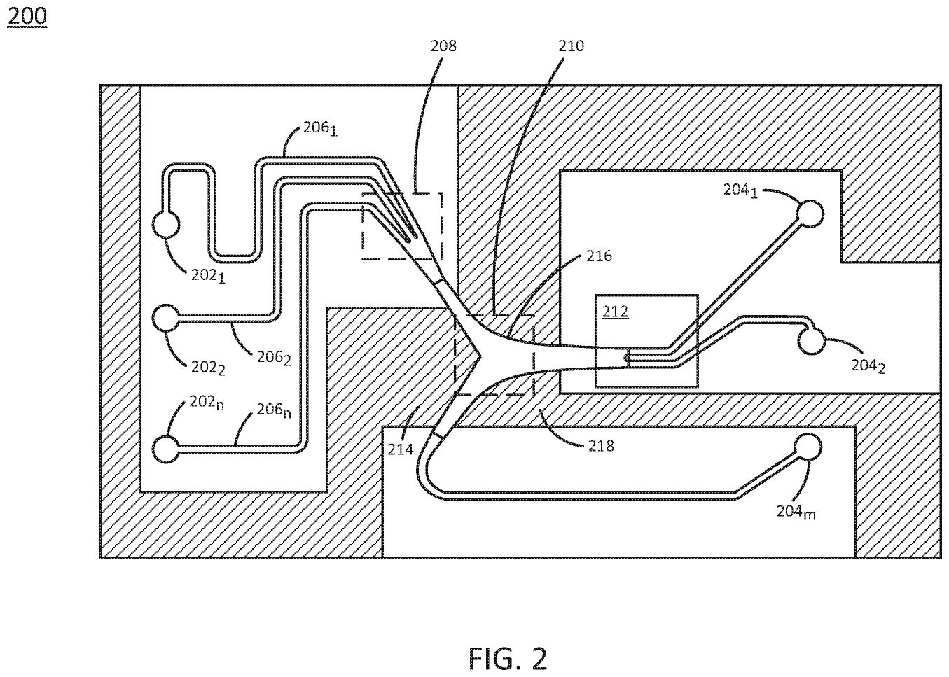

FIG. 2 illustrates a more detailed example of a microfluidic device 200. In one example, the microfluidic device 200 may include sample inlets 202₁-202ₙ (hereinafter also referred to individually as an inlet 202 or collectively as inlets 202). Each inlet 202 may be coupled to a microfluidic channel 206₁-206ₙ (hereinafter also referred to individually as a microfluidic channel 206 or collectively as microfluidic channels 206). In one example, a sheath fluid that carries different particles or cells may help the particles to generally line up while passing through a DEP separator 210. The sheath fluid may provide for a relatively easier separation of the particles in the DEP separator 210.

In one example, the sheath fluid may be a buffer solution that is compatible with the separations performed by the microfluidic device 200. For example, the buffer solution may have a low conductivity pH 7 buffer that is made to be isotonic to the cells via sucrose. For example, the buffer may be a solution of about 9.5% sucrose, about 0.1 milligram per milliliter (mg/mL) dextrose, about 0.1% pluronic F68, about 0.1% bovine serum albumin, about 1 millimolar (mM) phosphate buffer pH 7 (adjustable), about 0.1 mM CaAcetate, about 0.5 mM MgAcetate, and about 100 units/ml catalase.

In one example, the flow rate of the fluid may be greater than the flow rate of the particles. For example, the fluid may flow at about 0.2 mL/minute (min). In other examples, the flow rate may be increased to about 20 mL/min or decreased to about 0.001 mL/min. The flow rate of the fluid may be approximately 20 times the flow rate of the particles within the fluid.

The particles may be cells. The cells may include cells that are of interest and not of interest. The microfluidic device 200 may be used to separate the cells of interest from the remaining types of cells in the fluid. The cells may include red blood cells, white blood cells, platelets, cancer cells, bacteria, yeast, microorganisms, or any other type of biological microparticles including proteins. The fluid may include two or more different types of particles that are to be separated out. In one example, the fluid may include three or more different types of particles that are to be separated out (e.g., one particle separated out by the DEP separator 210 and a second particle separated out by a density separator 212).

In an example, the microfluidic channels 206 may merge into a single channel that flows through a focuser 208. The focuser 208 may be a region that is shaped to control a location of the particles in the fluid before the particles enter the DEP separator 210. For example, the focuser 208 may be a tapered section in the microfluidic channels 206 to narrow the cross-section of the channel in which the fluid flows.

In one example, the focuser 208 may be a dual axis focuser. In other words, the focuser 208 may control the location along a vertical axis and a horizontal axis. For example, the focuser 208 may taper the portion of the channel in both the vertical direction and the horizontal direction.

In one example, the focuser 208 may focus the particles in a center of the channel. In another example, the focuser 208 may focus the particles towards a top of a vertical axis, but in a center of the horizontal axis. As a result, the higher vertical location in the channel may help improve the downstream density separation, as discussed below.

In one example, the focuser 208 may focus the particles in the fluid as they move towards the DEP separator 210. The DEP separator 210 may implement an electrical field on the particles in order to force the particles to be separated from each other and pass into different outlet channels 204₁-204ₘ (hereinafter also referred to individually as a channel 204 or collectively as channels 204). In order for the electrical field to affect the plurality of particles or cells, the particles do not have to be charged. Instead, because particles such as cells exhibit dielectrophoretic activity in the presence of the electric field, the different particles may react differently in the presence of the electrical field and are, thereby, separated as they travel through the DEP separator 210.

In one example, the DEP separator 210 may include a first electrode 216, a second electrode 218 and a ground electrode 214. A voltage source or wave generator may apply a current or voltage through the first electrode 216 and the second electrode 218 to create an electrical field. In one example, the voltage source may be communicatively coupled to a controller or a processor that controls an amount of voltage or current that is passed through the first electrode 216 and the second electrode 218. The amount of voltage or current that is passed may depend on the type of particle or cells that are injected into the inlet channels 202. The DEP separator 210 may separate the particles along a horizontal plane (e.g., left (towards a top of the page) and right (towards a bottom of the page)).

In one example, some of the particles that are not part of the diagnostic process may be separated out by the DEP separator 210 into the outlet channel 204ₘ. In one example, the outlet channel 204ₘ may be a waste outlet channel. The remaining particles may continue towards the outlet channels 204₁ and 204₂.

In one example, the fluid containing the remaining particles for diagnosis may continue to flow towards the density separator 212. In one example, the density separator 212 may separate the particles along a vertical plane (e.g., into the page and out of the page). The density separator 212 may use buoyancy forces and the different densities of the particles to perform an additional separation downstream from the DEP separator 210.

In one example, the DEP separator 210 and the density separator 212 may be integrated into a single device. In other words, at least one of the microfluidic channels 102 may carry particles through both the DEP separator 210 and the density separator 212. In other words, at least some of the particles may be separated by both the DEP separator 210 and the density separator 212 in a single continuous process.

In one example, a length of the channel measured between an exit of the DEP separator 210 to the entrance of the density separator 212 may be long enough for the density separation between the particles to occur. The length of the channel may be based on a flowrate of the fluid, a viscosity of the fluid, an average radius of the plurality of different particles or cells in the fluid, a density of each one of the different particles or cells, and the like.

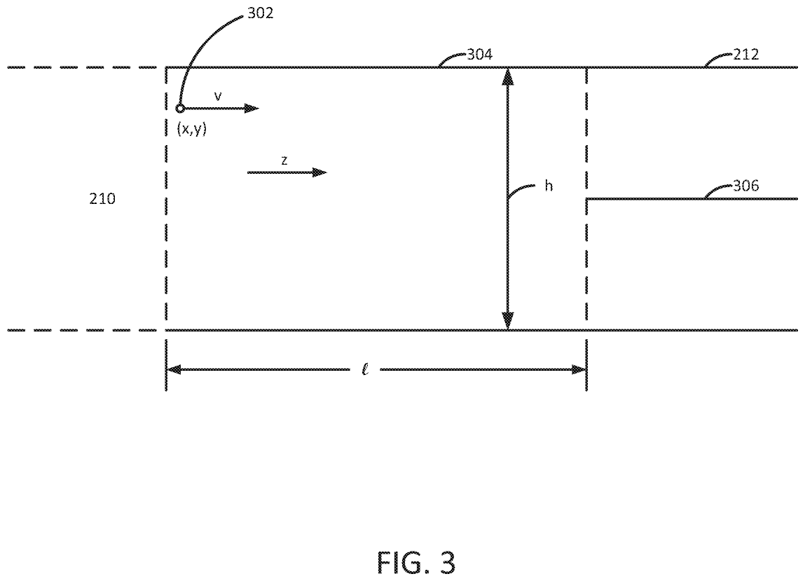

FIG. 3 illustrates an example of various parameters that are used to determine a length of a channel 304. In one example, a length “I” of the channel in the density separator 212 may be defined to be a distance between an exit of the DEP separator 210 and an entrance of the density separator 212. In one example, the exit of the DEP separator 210 may be a point where the particles are past the electrodes 216 and 218 or the electrical field generated by the electrodes 216 and 218.

The entrance of the density separator 212 may be a front edge of a plane 306 in the density separator 212. The plane 306 may be a physical divider within the microfluidic channel and may be made of the same material as the microfluidic channel. The plane 306 may divide the particles along a vertical axis. In other words, heavier or denser particles may flow below the plane 306 and lighter or less dense particles may flow above the plane 306. The plane 306 may be any type of material that is inserted into the microfluidic channels.

In one example, the length of the channel 304 may be based on a position (x,y) of a particle 302 along a height “h” of the channel and along an axial direction “z”. The length of the channel 304 may also be a function of a velocity “v” of the particle 302, a density of the particle 302, and a viscosity of the particle 302 and the fluid that contains the particle 302. In one example, the length of the channel may be defined in accordance with a function for y=h/2, x =0 in an x-y plane as follows:

$$l = \frac{G \text{/} 2 μ}{2 ( {ρ}_{p} - {ρ}_{f} ) g {r}^{2} \text{/} 9 μ} ( ( \frac{- {y}^{2} ( 2 y - 3 h )}{6} ) + \frac{{h}^{3}}{12} ) , \qquad ( EQN . 1 )$$

where G is proportional to a flowrate of the fluid containing the particle (p) 302 (and defined below), Q is the flow rate of the fluid containing the particle (p) 302, μ represents a viscosity of the fluid (f), y is a vertical position along a height (h) of the channel 304, g is a gravitational constant, r is an average radius of the particle 302, ρₚ is a density of the particle 302, and ρ₇₁ is a density of the fluid.

The function above may be based on the velocity of the particle in the vertical dimension moving away from the focuser 208 given by:

$$\frac{d y}{d t} = \frac{2 ( {ρ}_{p} - {ρ}_{f} ) g {r}^{2}}{9 μ} , \qquad ( EQN . 2 )$$

And the velocity of the particle in the horizontal dimension (or a position x along the axial direction z) given by:

$$\frac{d x}{d t} = \frac{G}{2 μ} y ( h - y ) , Q = \frac{G {h}^{3}}{12 μ} , \qquad ( EQN . 3 )$$

where μ(0)=μ(h)=0 as the velocity follows a parabolic profile.

The above functions are provided as example functions that can be used to measure the length of the channel 304 when the focuser 208 positions the particles in a center of the channel 304 (e.g., y=h/2 and x=0). However, the functions can be modified to calculate the length of the channel 304 for when the focuser 208 positions the particles towards a top of the channel 304 (e.g., y=h and x=0).

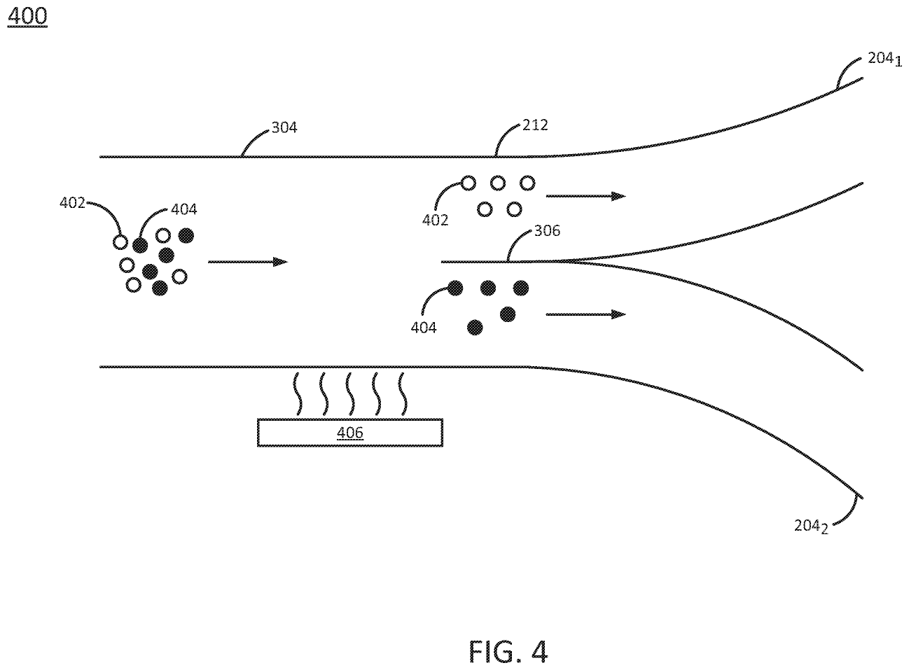

FIG. 4 illustrates an example of a schematic diagram 400 of a vertical separation of particles in the density separator 212. In one example, the fluid entering the density separator 212 may include particles 402 and 404. In one example, the density of the particles 404 may be greater than the density of the particles 402. However, the dielectrophoretic properties of the particles 402 and 404 may be similar. Thus, the particles 402 and 404 may remain together after the DEP separator 210.

In one example, the length of the channel 304 may be designed to be an appropriate length based on the parameters described above and discussed with respect to FIG. 3. As the particles 402 and 404 travel down the length of the channel 304, the particles 402 and 404 may separate in a vertical direction as shown in FIG. 4.

In one example, the lighter particles 402 may travel above the plane 306 and the heavier particles 404 may travel below the plane 306. The particles 402 may travel towards the outlet channel 204₁ and the particles 404 may travel towards the outlet channel 204₂.

In one example, a magnet 406 may be located in or adjacent to the channel 304 to assist the density separator 212. For example, a particle of interest (e.g., the particles 404) may be probed for using antibodies coated on magnetic beads. The magnet 406 may attract the magnetic beads as the particles 404 travel down the channel 304. As a result, the particles 404 may move towards the magnet 406 and may move down faster than would otherwise occur via natural gravitational forces within the channel 304. The magnet 406 may allow the length of the channel 304 to be shorter, thereby, reducing the overall size and costs of the microfluidic device 200.

In another example, the microfluidic device 200 may be placed in a centrifuge device. The centrifuge device may apply a centrifugal force on the microfluidic device 200. The centrifugal force may be applied in a same direction as a desired separation direction in the density separator 212. Thus, the centrifugal force may also allow the length of the channel 304 to be shorter, thereby, reducing the overall size and costs of the microfluidic device 200.

Although only a single DEP separator 210 and a single density separator 212 are illustrated in FIG. 2, it should be noted that the microfluidic device 200 may include any number of DEP separators 210 and density separators 212. For example, a series of DEP separators 210 may be used and a series of density separators 212 may be used downstream from the DEP separators 210. In one example, the microfluidic device 200 may include a plurality of different vertical layers. Thus, multiple density separators 212 may be used to separate particles into respective layers of the plurality of different vertical layers.

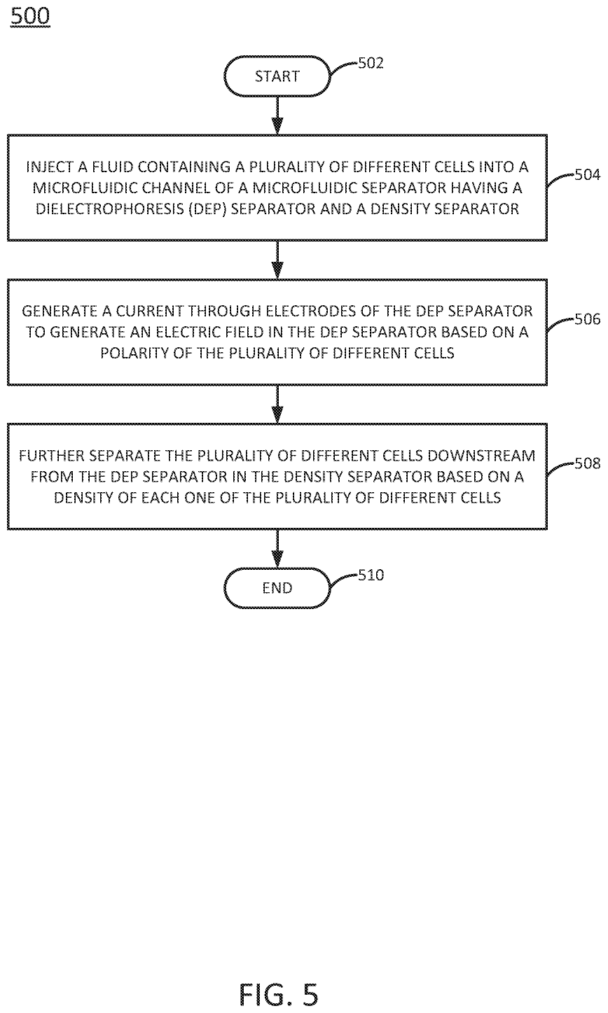

FIG. 5 illustrates a flow diagram of an example method 500 for separating particles. In an example, the method 500 may be performed by the microfluidic device 100, 200, or 300 described above.

At block 502, the method 500 begins. At block 504, the method 500 injects a fluid containing a plurality of different cells into a microfluidic channel of a microfluidic separator having a dielectrophoresis (DEP) separator and a density separator. In one example, the fluid may be a buffer solution that carries the different cells in a solution in the microfluidic channel.

In one example, each cell may be injected into a different microfluidic inlet. The fluids carrying the cells may be combined before entering a focusing region. The focusing region may control where in the microfluidic channel that the different types of cells may enter the DEP separator region. The focusing region may include a dual axis focuser. In other words, the focusing region may focus where the different types of cells are located in a horizontal plane and a vertical plane in the microfluidic channel

In one example, the focusing region may focus the fluid with the different types of cells towards a top of the microfluidic channel. As a result, gravity may further assist in the separation of the cells based on the density of the cells as the cells move towards the density separator.

At block 506, the method 500 generates a current through electrodes of the DEP separator to generate an electric field in the DEP separator based on a polarity of the plurality of different cells. As described above, the DEP separator may include a ground electrode, a positive electrode, and a negative electrode. A voltage may be applied across electrodes to generate an electric field. The different cells may have different dielectrophoresis properties. The cells may be separated along a horizontal plane (e.g., left and right) in the DEP separator based on a polarity of each cell as the cells move through the electric field in the DEP separator.

At block 508, the method 500 further separates the plurality of different cells downstream from the DEP separator in the density separator based on a density of each one of the plurality of different cells. In one example, the density separator may be used to further separate cells downstream from the DEP separator. For example, the fluid may contain three different types of cells. One type of cell may be separated by the DEP separator. The remaining two types of cells may be separated by the density separator.

In one example, the length of the microfluidic channel from the focusing region to the density separator may be long enough to allow the cells to separate based on density as the cells move down the microfluidic channel, as described above. The density separator may then separate the two types of cells in a vertical plane (e.g., up and down).

In one example, a type of cell of interest of the plurality of different cells may be probed for using antibodies coated on a magnetic bead. A magnet or magnetic force may be applied in the density separator. The magnetic force may attract the type of cell of interest through attracting the magnetic bead coated with antibodies specific to the cell to further improve the density separation. In another example, the microfluidic device may be placed in a centrifuge. The centrifugal force generated by the rotation of the centrifuge may be used to assist the density separation. The separated cells may flow towards respective outlets of the microfluidic device. At block 510, the method 500 ends.

It will be appreciated that variants of the above-disclosed and other features and functions, or alternatives thereof, may be combined into many other different systems or applications. Various presently unforeseen or unanticipated alternatives, modifications, variations, or improvements therein may be subsequently made by those skilled in the art which are also intended to be encompassed by the following claims.

1. An apparatus, comprising:
a microfluidic channel to receive a fluid containing a plurality of different cells;
a dielectrophoresis (DEP) separator to separate the plurality of different cells passing through the DEP separator within the microfluidic channel; and
a density separator to further separate a portion of the plurality of different cells from the DEP separator based on a density of each one of the plurality of different cells.

2. The apparatus of claim 1, further comprising:
a plurality of outlet channels downstream of the microfluidic channel to collect each different type of cell of the plurality of different cells that is separated out by the DEP separator and the density separator.

3. The apparatus of claim 1, wherein the DEP separator comprises a plurality of electrodes to generate an electric field.

4. The apparatus of claim 1, wherein the DEP separator separates the plurality of different cells in a horizontal direction.

5. The apparatus of claim 1, wherein a length of a channel in the density separator is based on a flowrate of the fluid, a viscosity of the fluid, a radius of the plurality of different cells, and the density of the each one of the plurality of different cells.

6. The apparatus of claim 1, wherein density separator comprises a separation plane to separate the plurality of different cells in a vertical direction.

7. An apparatus, comprising:
a microfluidic channel to receive a fluid containing a plurality of different cells;
a focuser to control a location of the plurality of different cells within the microfluidic channel;
a dielectrophoresis (DEP) separator to separate the plurality of different cells passing through DEP separator within the microfluidic channel; and
a density separator downstream from the DEP separator to further separate the plurality of different cells based on a density of each one of the plurality of different cells.

8. The apparatus of claim 7, wherein the focuser provides dual axis focusing.

9. The apparatus of claim 8, wherein the focuser is to bias the plurality of different cells above a center of the microfluidic channel.

10. The apparatus of claim 7, further comprising:
a plurality of outlet channels downstream of the microfluidic channel at different vertical planes to collect each different type of cell of the plurality of different cells that is separated out by the DEP separator and the density separator.

11. The apparatus of claim 7, wherein the density separator further comprises:
a magnet to assist in a vertical separation of the plurality of different cells in the density separator.

12. The apparatus of claim 7, further comprising:
a centrifuge to hold the apparatus, wherein the centrifuge is to apply a centrifugal force to assist the separation of the plurality of different cells in the density separator.

13. A method, comprising:
injecting a fluid containing a plurality of different cells into a microfluidic channel of a microfluidic separator having a dielectrophoresis (DEP) separator and a density separator;
generating a current through electrodes of the DEP separator to generate an electric field in the DEP separator based on a polarity of the plurality of different cells; and
further separating the plurality of different cells downstream from the DEP separator in the density separator based on a density of each one of the plurality of different cells.

14. The method of claim 13, further comprising:
focusing the fluid towards a top of the microfluidic channel to assist the density separator.

15. The method of claim 13, further comprising:
probing a type of cell of interest of the plurality of different cells with antibodies coated on a magnetic bead; and
applying a magnetic force in the density separator to separate the type of cell of interest in the density separator.
